(19) United States
(12) Patent Application Publication
(10) Pub. No.: US 2025/0089863 A1
(43) Pub. Date: Mar. 20, 2025

(54) TECHNIQUES FOR FIXING AN OUTER COVER TO A WEARABLE RING DEVICE

(71) Applicant: Oura Health Oy, Oulu (FI)

(72) Inventors: Teemu Juhani Haverinen, Oulu (FI); Heikki Juhani Huttunen, Haukipudas (FI); Sami Sakari Ihme, Oulu (FI); Jouni Juhani Huopana, Oulu (FI); Marko Uusitalo, Oulu (FI); Antti Kalevi Lämsä, Oulu (FI); Kari Kuisma Kanniainen, Ii (FI)

(21) Appl. No.: 18/747,128

(22) Filed: Jun. 18, 2024

Related U.S. Application Data
(63) Continuation-in-part of application No. 18/471,143, filed on Sep. 20, 2023.

(51) Int. Cl. A44C 9/00

(52) U.S. Cl. CPC A44C 9/0053

(57) ABSTRACT

Methods, systems, and devices for manufacturing a wearable ring device are described. An outer cover may be placed around a ring assembly, including a printed circuit board (PCB) coupled to an inner cover, producing a first slot between the outer cover and the inner cover on a first lateral side of the wearable ring device and a second slot between the between the outer cover and the inner cover on a second lateral side of the wearable ring device. Additionally, a first and second side cover may be inserted into the first and second slots, respectively, where the insertion causes the first and second side covers to mechanically deform. In such cases, the mechanical deformation may cause the first and second side covers to engage one or more locking mechanisms of the inner cover, the outer cover, or both, to secure the inner cover to the outer cover.

### CROSS REFERENCE

The present Application for Patent is a continuation-in-part of U.S. Provisional Patent Application No. 18/471,143 by HAVERINEN et al., entitled “TECHNIQUES FOR FIXING AN OUTER COVER TO A WEARABLE RING DEVICE,” filed Sep. 20, 2023, assigned to the assignee hereof, and expressly incorporated by reference herein.

### FIELD OF TECHNOLOGY

The following relates to wearable devices and data processing, including techniques for fixing an outer cover to a wearable ring device.

### BACKGROUND

Some wearable devices may be configured to collect data from users to help the users understand more about their overall physiological health and well-being. However, wearable devices may be exposed to external forces while worn by the user, which may cause one or more components of the wearable devices to become loose or move in an unintentional manner, reducing a lifespan of the wearable device.

### BRIEF DESCRIPTION OF THE DRAWINGS

FIG. 1 illustrates an example of a system that supports techniques for fixing an outer cover to a wearable ring device in accordance with aspects of the present disclosure.

FIG. 2 illustrates an example of a system that supports techniques for fixing an outer cover to a wearable ring device in accordance with aspects of the present disclosure.

FIG. 3 shows an example of a system that supports techniques for fixing an outer cover to a wearable ring device in accordance with aspects of the present disclosure.

FIGS. 4A, 4B, and 4C show example cross-sectional views of a wearable ring device that supports techniques for fixing an outer cover to a wearable ring device in accordance with aspects of the present disclosure.

FIG. 5 shows a flowchart illustrating methods that support techniques for fixing an outer cover to a wearable ring device in accordance with aspects of the present disclosure.

### DETAILED DESCRIPTION

Some wearable devices, such as wearable ring devices, include a metal outer cover and an epoxy inner cover. As such, manufacturing techniques for the wearable ring devices may manufacture the wearable ring device from the “outside in,” such that electrical components of the wearable ring device, such as a printed circuit board (PCB) and one or more optical sensors, are manufactured within an outer metal cover, or shell, and secured with an inner epoxy cover, or shell, to form the wearable ring device. That is, the outer cover, with the electrical components, is placed into a mold, and the inner epoxy cover is formed to the outer cover via injection molding. However, such manufacturing techniques suffer from several technical shortfalls, including requiring tedious manual alignment of the optical components of the PCB within the mold by operators, requiring post-manufacturing polishing that risks damaging the wearable ring device, and demonstrating an inability to efficiently support different wearable ring device covers.

As such, as compared to wearable ring devices that are manufactured “outside in” (e.g., from outer cover to inner cover), a wearable ring device may be manufactured “inside out.” Specifically, the electrical components (e.g., the PCB and one or more optical sensors) may be attached to an inner metal shell of the wearable ring device and the inner shell (e.g., and attached electrical components) may be placed into a mold, such that a clear epoxy may be injection molded to secure the electrical components to the inner shell. The injection molding may further fill one or more apertures of the inner shell with the clear epoxy, such that the one or more optical sensors may be secured with relation to the one or more apertures to enable data collection. The result of injection molding the inner shell to the electrical components may be referred to as an inner cover, or a ring engine assembly, that is essentially an operational ring without an outer cover. Subsequently, different outer covers may be slid around the inner cover and may be secured to the inner cover using side covers, to finish the ring. However, methods for securing the side covers to the outer cover and the inner cover may be deficient, resulting in the outer cover inadvertently rotating around the inner cover, the side covers coming loose or popping off the wearable ring device, or the like.

Accordingly, techniques described herein may support a manufacturing process for wearable ring devices to secure an outer cover of a wearable ring device to the inner cover of the wearable ring device, reducing unintentional movement of the side covers, the outer cover, or both, when exposed to an external force. In particular, the outer cover may be slid around the inner cover (e.g., ring engine assembly) and side covers (e.g., ring-shaped fittings) may be placed on each side of the ring, aligning with respective slots between the outer cover and the inner cover on each side of the ring. Each of the side covers may be slightly wider than the slots between the outer cover and the inner cover, such that the side covers mechanically deform upon being inserted into the slots due to an applied force (e.g., upon being pressed into the slots). Additionally, the outer cover, the inner cover, or both, may include one or more mechanical locking features, or grooves, positioned at least partially within the slots on either side of the ring, such that the mechanical deformation of the side covers causes the side covers to engage, or fill, the mechanical locking features within the outer cover, the inner cover, or both, thereby locking the outer cover to the inner cover. In some cases, the side covers may be easily removed and replaced, such that multiple outer covers may be supported by the wearable ring device and may be interchangeable. Additionally, or alternatively, heat may be applied to the side covers, such that the side covers permanently deform under the applied heat to more closely engage, or fill, the mechanical locking features.

Additionally, or alternatively, the side covers may include one or more flanges, or locking wings, to enable the side covers to engage, or fill, the mechanical locking features within the outer cover, the inner cover, or both. In other words, each of the side covers may be the same width as the slots (e.g., or within a threshold tolerance), with exception to a flange on the inner radius of the side cover, a flange on the outer radius of the side cover, or both. As such, when the side covers are inserted into the slots due to an applied force (e.g., pressed into the slots), the flanges may deform, or compress inwards, as they are pushed into the slot and may retake their original shape, or “pop out,” when they reach, or enter, the mechanical locking features on the outer cover, the inner cover, or both, thereby locking the outer cover to the inner cover. In such cases, friction created between the outer cover and the inner cover may keep the outer cover from sliding around the inner cover, as well as keep the side covers from popping out of the slot.

Additionally, or alternatively, a binding agent may be applied between the outer cover and the inner cover to lock, or secure, the outer cover to the inner cover. For example, a light sensitive glue, such as ultra-violet (UV) glue, may be applied to the outer cover and the inner cover, such that the slots (e.g., as well as any gaps between the inner cover and the outer cover) are at least partially filled or coated with the UV glue. Additionally, the UV glue may be exposed to UV light (e.g., directly or through the side covers), such that the UV glue cures and becomes solid, thereby locking the outer cover to the inner cover. In some other examples, heat activated film (HAF) may be applied to the outer cover, the inner cover, or both, such that when the outer cover is placed around the inner cover, the HAF contacts both the outer cover and the inner cover. Additionally, the HAF may be exposed to heat through the outer cover, the inner cover, the side covers, or any combination thereof, such that the HAF is activated, thereby locking the outer cover to the inner cover.

Additionally, or alternatively, the texture of the outer cover, the inner cover, or both, may be modified, such that friction between the side covers and the outer cover, the inner cover, or both, is increased (e.g., as compared to an unmodified texture). For example, portions of the outer cover that align with the slots, portions of the inner cover that align with the slots, or both, may be laser engraved, sand blasted, sanded, or the like thereof, to increase a coefficient of friction between the side covers and the outer cover, the inner cover, or both, within the slots (e.g., relative to unmodified components). As such, a force required to cause the outer cover to rotate around the inner cover, the side covers to come loose or pop off the wearable ring device, or both, may increase to a value that is unlikely to be experienced by the wearable ring device while the wearable ring device is worn by the user.

Aspects of the disclosure are initially described in the context of systems supporting physiological data collection from users via wearable devices. Aspects of the disclosure are further illustrated in cross-sectional views of a wearable ring device Aspects of the disclosure are further illustrated by and described with reference to apparatus diagrams, system diagrams, and flowcharts that relate to techniques for fixing an outer cover to a wearable ring device.

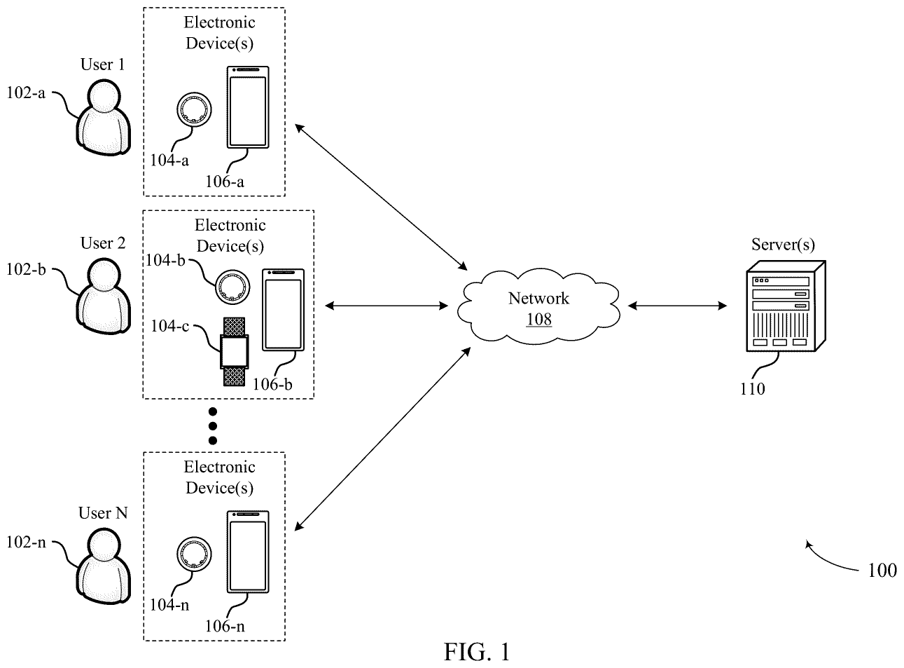

FIG. 1 illustrates an example of a system 100 that supports techniques for fixing an outer cover to a wearable ring device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may be manufactured according to the techniques described herein. For example, each ring 104 may include an inner cover and an outer cover. In some cases, electrical components (e.g., PCB, optical sensors, etc.) may be secured or otherwise attached to the inner cover/shell of the ring, where the inner cover/shell and the electrical components may collectively be referred to as a “ring engine assembly,” which is essentially a completed ring without an outer cover. In some cases, the electrical components may be secured to the inner metal shell via injection molding using a clear epoxy, thereby forming the ring engine assembly.

Subsequently, the outer cover may be placed or slid around the inner cover, and side covers (e.g., ring-shaped fittings) may be placed on each side of the ring 104, aligning with respective slots between the outer cover and the inner cover on each side of the ring 104. Each of the side covers may be slightly wider than the slots between the outer cover and the inner cover, such that the side covers mechanically deform upon being inserted into the slots due to an applied force (e.g., upon being pressed into the slots). Additionally, the outer cover, the inner cover, or both, may include one or more mechanical locking features, or grooves, positioned at least partially within the slots on either side of the ring 104, such that the mechanical deformation of the side covers causes the side covers to engage, or fill, the mechanical locking features within the outer cover, the inner cover, or both, thereby locking the outer cover to the inner cover. In some cases, the side covers may be easily removed and replaced, such that multiple outer covers may be supported by the ring 104 and may be interchangeable. For example, a user 102 may be able to insert a removal tool at least partially into a slot including a side cover, such that the removal tool “pops” the side cover out from between the outer cover and inner cover. As such, the user 102 may remove both side covers and replace the outer cover with a new outer cover, selected from multiple outer covers that are compatible with the ring 104.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

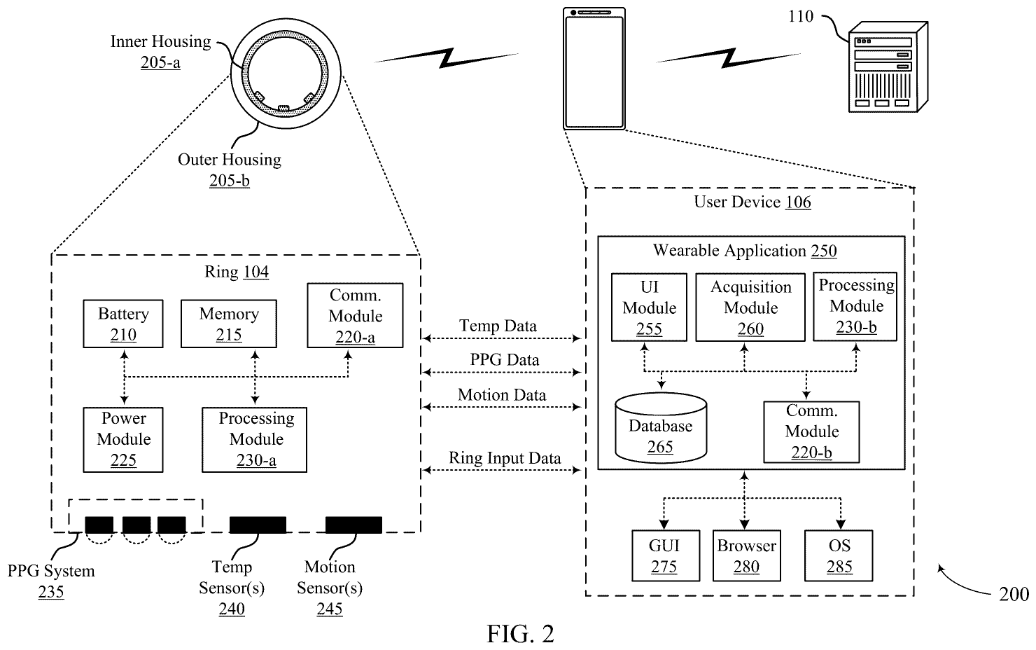

FIG. 2 illustrates an example of a system 200 that supports techniques for fixing an outer cover to a wearable ring device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more PCBs, such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS) 285, a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support methods for manufacturing the ring 104 in accordance with the techniques described herein. For example, electrical components of the ring 104, such as the battery 210, the memory 215, the communication module 220-a, the power module 225, the processing module 230-a, the PPG system 235, the temperature sensor(s) 240, the motion sensor(s) 245, or any combination thereof, may be attached to an inner metal cover, or shell. Additionally, the inner metal cover, attached to the electrical components, may be placed in a mold, such that a clear epoxy can be injection molded to secure the electrical components to the inner metal cover. The injection molding may further fill one or more apertures of the inner metal cover with the clear epoxy, such that the one or more optical sensors, such as the PPG system 235, may be secured with relation to the one or more apertures to enable data collection through the one or more apertures. The result of injection molding the inner metal cover to the electrical components may be referred to as the inner housing 205-a, or engine assembly, of the ring 104, which is essentially an operational ring 104 without the outer housing 205-b.

Subsequently, the outer housing 205-b (e.g., an outer cover) may be slid, or placed, around the inner housing 205-a and may be secured to the inner housing 205-a using side covers (e.g., ring-shaped fittings). That is, side covers may be placed on each side of the ring 104, aligning with respective slots between the outer housing 205-b and the inner housing 205-a on each side of the ring 104. Each of the side covers may be slightly wider than the slots between the outer housing 205-b and the inner housing 205-a, such that the side covers mechanically deform upon being inserted into the slots due to an applied force (e.g., upon being pressed into the slots). Additionally, the outer housing 205-b, the inner housing 205-a, or both, may include one or more mechanical locking features, or grooves, positioned at least partially within the slots on either side of the ring 104, such that the mechanical deformation of the side covers causes the side covers to engage, or fill, the mechanical locking features within the outer housing 205-b, the inner housing 205-a, or both, thereby locking the outer housing 205-b to the inner housing 205-a.

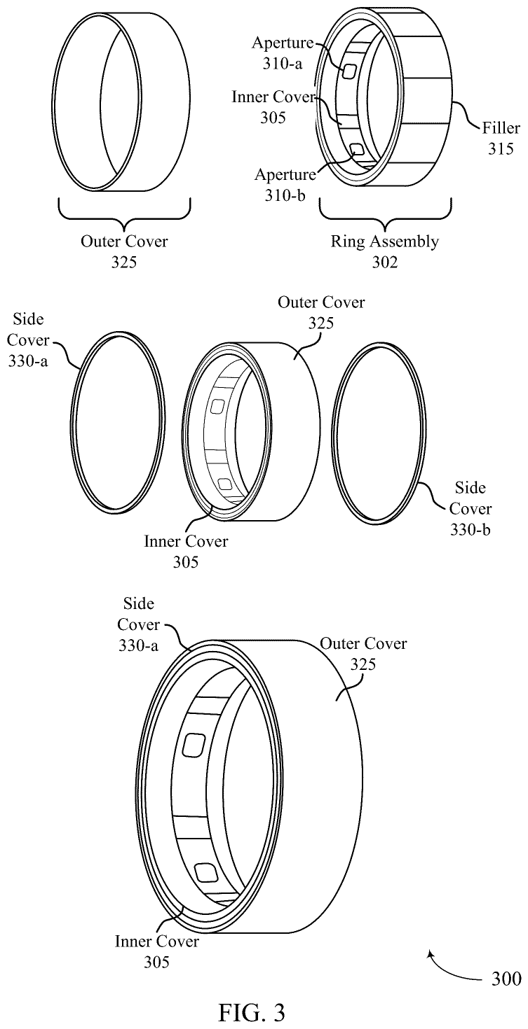

FIG. 3 shows an example of a system 300 that supports techniques for fixing an outer cover to a wearable ring device in accordance with aspects of the present disclosure. In other words, the system 300 may support techniques for a ring 104 to be manufactured “inside out,” securing an outer cover 325 to an inner cover 305, including electrical components of the ring 104, via multiple side covers 330, in a manner that reduces unintentional movement of the side covers 330, the outer cover 325, or both when exposed to an external force.

For example, the electrical components of the ring 104 (e.g., the PCB and one or more optical sensors) may be attached to an inner cover 305 (e.g., inner ring-shaped housing) of the ring 104 and the inner cover 305 (e.g., and attached electrical components) may be placed into a mold, such that a filler 315, such as a clear epoxy, may be injected into the mold to secure the electrical components to the inner cover 305. Additionally, the injection molding may cause the filler 315 to fill one or more apertures 310 of the inner cover 305, such as an aperture 310-a and an aperture 310-b, such that the one or more optical sensors may be secured with relation to the apertures 310 to enable data collection. The result of injection molding the inner cover 305 to the electrical components may be referred to as a ring assembly 302 (e.g., ring engine assembly). The ring assembly 302 may essentially be an operational ring 104 without an outer cover 325 (e.g., outer ring-shaped housing). Subsequently, different outer covers 325 may be slid around the inner cover 305 (e.g., ring assembly 302) and may be secured to the inner cover 305 using side covers 330, such as the side cover 330-a and the side cover 330-b, to finish the ring. However, methods for securing the side covers 330 to the outer cover 325 and the inner cover 305 may be deficient, resulting in the outer cover 325 rotating around the inner cover 305, the side covers 330 coming loose or popping off the ring 104, or both.

Accordingly, techniques described herein may support a manufacturing process for rings 104 to secure the outer cover 325 to the inner cover 305 (e.g. ring assembly 302) in a manner that reduces or eliminates unintentional movement of the side covers 330, the outer cover 325, or both, when exposed to an external force. In particular, the outer cover 325 may be slid around the inner cover 305 (e.g., ring assembly 302) and side covers 330 (e.g., ring-shaped fittings) may be placed on each side of the ring 104, aligning with respective slots between the outer cover 325 and the inner cover 305 on each side of the ring 104. For example, the side cover 330-a may be placed on a first lateral side of the ring 104 aligning with a first slot between the outer cover 325 and the inner cover 305 (e.g., on the first lateral side) and the side cover 330-b may be placed on a second lateral side of the ring 104, opposite the first lateral side, aligning with a second slot between the outer cover 325 and the inner cover 305 (e.g., on the second lateral side). As such, the side cover 330-a may be inserted into the first slot and the side cover 330-b may be inserted into the second slot.

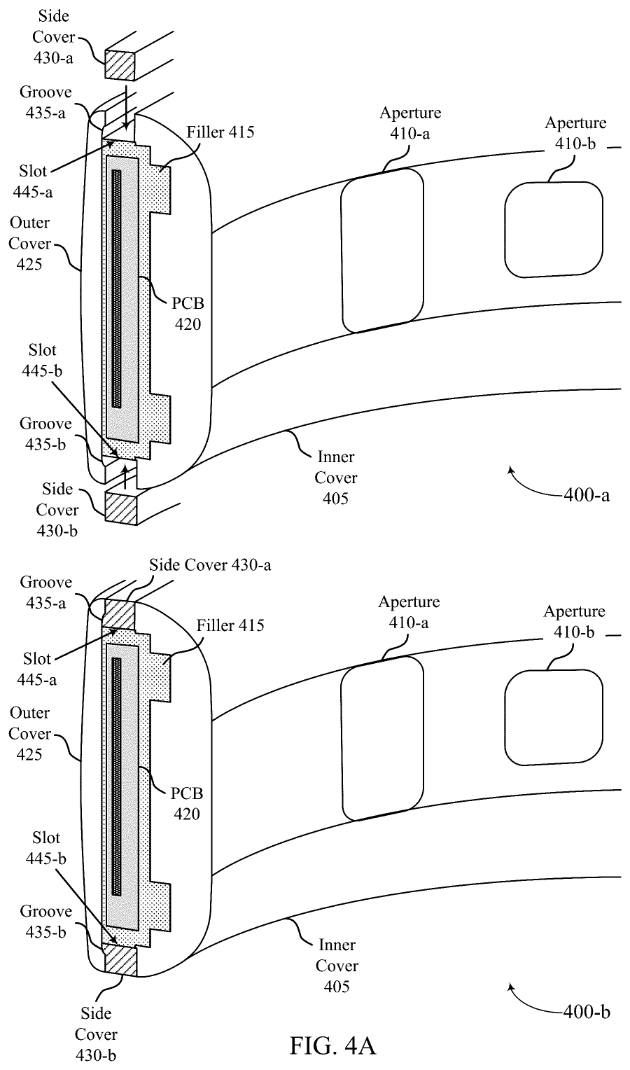

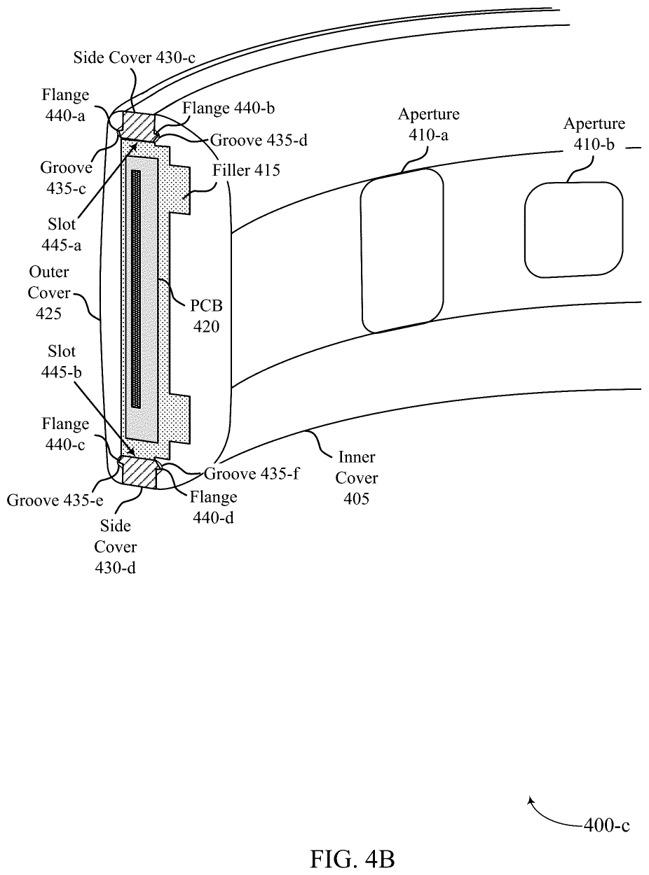

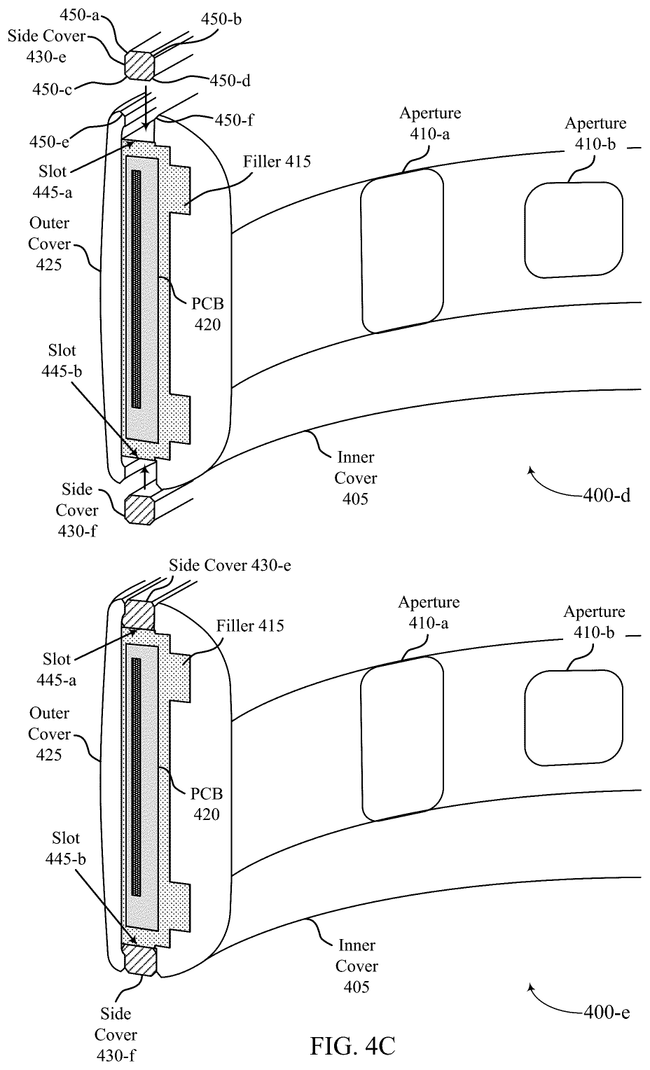

In some examples, each of the side covers 330 may be slightly wider than the slots between the outer cover 325 and the inner cover 305, such that the side covers 330 mechanically deform upon being inserted, or pressed, into the slots due to an applied force. In such cases, the mechanical deformation of the side covers 330 may cause the side covers 330 to engage with one or more mechanical locking features on the outer cover 325, the inner cover 305, or both, as described further with reference to FIG. 4A. Additionally, in some cases, each of the side covers 330 may include at least a first set of chamfers, where the first set of chamfers are configured to contact a second set of chamfers on the outer cover 325, the inner cover 305, or both, to guide the side covers 330 into the slots (e.g., while the slots are inserted into the slots), as described with reference to FIG. 4C.

Additionally, or alternatively, each of the side covers 330 may include one or more flanges, or locking wings, to enable the side covers 330 to engage the one or more mechanical locking features on the outer cover 325, the inner cover 305, or both, as described further with reference to FIG. 4B.

In some examples, a binding agent may be applied, or dispensed, between the outer cover 325 and the inner cover 305 to lock, or secure, the outer cover 325 to the inner cover 305. For example, a heat or light sensitive glue (e.g., UV glue) may be applied to the slots (e.g., and any gaps) between the outer cover 325 and the inner cover 305, such that a thin layer of glue exists between the side covers 330 and the outer cover 325 and inner cover 305. In the case of light sensitive glues, the side covers 330 may be made of a light transparent material, such that light (e.g., UV light) may be applied to the glue through the sides covers 330 to cure the glue. Similarly, in the case of heat sensitive glue, heat may be applied to the glue through the side covers 330, the outer cover 325, the inner cover 305, or any combination thereof, to cure the glue. Curing the glue may cause the glue to solidify, thereby locking the outer cover 325 to the inner cover 305 (e.g., and locking the side covers 330 in place).

In some cases, heat or light sensitive glue may be used in place of side covers 330. For example, UV glue may be applied to the slots (e.g., and any gaps) between the outer cover 325 and the inner cover 305, such that the UV glue fills the entirety of the slots. In such cases, the UV light may be applied directly to the UV glue to cure the UV glue, such that the UV glue solidifies in place of the side covers 330, thereby locking the outer cover 325 to the inner cover 305. In additional or alternative implementations, a self-curing glue or composite may also be used. Such self-curing materials may be cured by mixing components that enable polymerization, or through exposure to air.

In some other examples, HAF may be applied to an inside surface of the outer cover 325, an outside surface of the inner cover 305, or both, such that when the outer cover 325 is placed around the inner cover 305, the HAF contacts both the outer cover 325 and the inner cover 305. Additionally, the HAF may be exposed to heat through the outer cover 325, the inner cover 305, the side covers 330, or any combination thereof, such that the HAF is activated, thereby locking the outer cover 325 to the inner cover 305.

Additionally, or alternatively, a texture of the outer cover 325, the inner cover 305, or both, may be modified, such that friction between the side covers 330 and the outer cover 325, the inner cover 305, or both, is increased (e.g., as compared to an unmodified texture). For example, one or more portions of the outer cover 325 that align with the slots, one or more portions of the inner cover 305 that align with the slots, or both, may be laser engraved, sand blasted, sanded (e.g., using grit paper), or the like, to increase a coefficient of friction (e.g., create a high friction locking mechanism) between the side cover 330 and the one or more portions of the outer cover 325, the one or more portions of the inner cover 305, or both, within the slots (e.g., relative to unmodified components). As such, after inserting the side covers 330 into the slots, a force required to cause the outer cover 325 to rotate around the inner cover 305, to cause the side covers 330 to come loose or pop off the wearable ring device, or both, may increase to meet or exceed a threshold value, where the threshold value based on external forces exerted on the ring 104 while being worn by a user 102 (e.g., the threshold value is greater than a maximum force likely to be exerted on the ring 104 while worn by the user 102).

In some implementations, light-emitting components (e.g., LEDs) may extend from the filler 315 of the ring assembly 302, and the side covers 330-a, 330-b may be at least partially transparent to light, such that light emitted by the light-emitting components may be seen through the side covers 330. In such cases, the light emitting components may be configured to emit different colors of light or strobe in different patterns to convey information to the user through the side covers 330. For example, the side covers 330 may be illuminated with different colors or patterns of light (e.g., strobing patterns) to indicate a battery level of the ring 104, to confirm receipt of a ring-inputted command or gesture, or the like.

FIGS. 4A, 4B, and 4C show example cross sectional views 400 (e.g., a cross-sectional view 400-a, a cross-sectional view 400-b, a cross-sectional view 400-c, a cross sectional view 400-d, a cross-sectional view 400-e) of a ring 104 that supports techniques for fixing an outer cover to a ring 104 in accordance with aspects of the present disclosure.

In some examples, as described with reference to FIG. 3, side covers 430 (e.g., side covers 330) may be inserted, or pressed, into slots between an outer cover 425 (e.g., an outer cover 325) and an inner cover 405 (e.g., an inner cover 305) to secure the outer cover 425 to the inner cover 405 in a manner that reduces unintentional movement of the side covers 430, the outer cover 425, or both, when exposed to an external force. For example, as described in the context of FIGS. 4A, 4B, and 4C, an inner cover 405 (e.g., ring engine assembly) may be manufactured by attaching electrical components of the ring 104, such as a PCB 420 and one or more optical sensors (e.g., among other electrical components), to an inner metal shell (e.g., inner ring-shaped housing) and placing the inner metal shell and attached electrical components in a mold. As such, filler 415 may be injected into the mold, such that the electrical components of the ring 104 are secured to the inner metal shell. Additionally, injecting filler 415 into the mold may result in the filler 415 filling one or more apertures 410 of the inner metal shell (e.g., of the ring 104), such as an aperture 410-a and an aperture 410-b, such that the one or more optical sensors, aligned with the apertures 410, may collect data (e.g., physiological data) via transmission of signaling (e.g., light) through the apertures 410. As described previously, the component produced as a result of the injection molding process (e.g., the inner metal shell molded to the electrical components via the filler 415) may be referred to as a ring assembly 302 (e.g., ring engine assembly).

As such, the outer cover 425 (e.g., outer ring-shaped housing) may be placed around the inner cover 405, producing a slot 445-a between the outer cover 425 and the inner cover 405 on a first lateral side of the ring 104 and a slot 445-b between the outer cover 425 and the inner cover 405 on a second lateral side of the ring 104, opposite the first lateral side. In such cases, a side cover 430 may be inserted into each slot 445. To insert the side covers 430 into the slots 445, a side cover 430 may be aligned with the slot 445 on the first lateral side of the ring 104 and the inner cover 405, the outer cover 425, and the aligned side cover 430 may be placed between two uniform surfaces, or plates, and placed into a press (e.g., machine press). As such, the press may apply a force (e.g., distributed force) to the uniform surfaces, transferring at least a portion of the force to the side cover 430 in a uniform manner, thus pressing (e.g., press-fitting) the side cover 430 into the slot 445. For example, the press may apply a distributed force along the circle-face surfaces (e.g., circular edges) of the ring 104, thereby forcing the side covers 430 into the respective slots 445. The process may then be repeated on the second lateral side of the ring 104 (e.g., or performed simultaneously with the first lateral side). In such cases, the side covers 430 may mechanically deform during (e.g., and after) insertion of the side covers 430 into the slots 445, such that the side covers 430 engage with one or more grooves 435 in the outer cover 425, the inner cover 405, or both, thereby securing the outer cover 425 to the inner cover 405 through “locking” of the side covers 430.

For example, as described with reference to the cross-sectional view 400-a, a side cover 430-a may be inserted, or pressed, into the slot 445-a and a side cover 430-b may be inserted, or pressed, into the slot 445-b. In such cases, the side cover 430-a and the side cover 430-b may be slightly wider than the slot 445-a and the slot 445-b, respectively, such that pressing, or inserting, the side covers 430 into the slots 445 cause the side covers 430 to mechanically deform. In other words, the side covers 430 may “squeeze” into the slots 445 and may engage grooves 435 in the outer cover 425, the inner cover 405, or both. For example, the outer cover 425 may include a groove 435-a and a groove 435-b, where each groove 435 is positioned at least partially within the slot 445-a and the slot 445-b, respectively. That is, each slot 445 may be of a first width for a portion of the slot 445 that does not include a groove 435 and may be of a second width for a portion of the slot 445 that includes the groove 435, where the second width is wider, or greater, than the first width. As such, after (e.g., and while) being inserted into the slots 445, as depicted in the cross-sectional view 400-b, the side covers 430 (which may exhibit a third width that is wider than both the first and second widths) may deform from the third width to the first width in the portion of the slot 445 that does not include the groove 435 and deform from the third width to the second width in the portion of the slot 445 that includes the groove 435, thus filling, or engaging, the groove 435.

As such, deformation of the side covers 430 from the third width to a width smaller than the third width (e.g., the first width and the second width) may result in generation of a force between the side covers 430 and the outer cover 425 and the inner cover 405, securing the side covers 430 in place between the outer cover 425 and the inner cover 405. Additionally, deformation of the side covers 430 into the groove 435-a and the groove 435-b may lock the side covers 430 in the slots 445, such that the side covers 430 may not “pop out” or release from the slots 445.

Additionally, or alternatively, as described with reference to the cross-sectional view 400-c in FIG. 4B, a side cover 430-c may be inserted, or pressed, into the slot 445-a and a side cover 430-d may be inserted, or pressed, into the slot 445-b. In such cases, the side cover 430-c and the side cover 430-d may each include one or more flanges 440. For example, the side cover 430-c may include a flange 440-a on an outer circumference (e.g., surface) of the side cover 430-c and a flange 440-b on an inner circumference of the side cover 430-c. Similarly, the side cover 430-d may include a flange 440-c on the outer circumference of the side cover 430-d and a flange 440-d on the inner circumference of the side cover 430-d. The flanges 430 may also be referred to as “locking wings.”

In some cases, a first portion of the side cover 430-c and the side cover 430-d that does not include the flanges 440 may be equal to a width of the slots 445, and a second portion of the side cover 430-c and the side cover 430-d that includes the flanges 440 may be wider than the slots 445. In other words, the flanges 440 may extend past a width of the slots 445. As such, while the side cover 430-c and the side cover 430-d are being inserted into the slots 445, the flanges 440 may deform, or depress, such that the side covers 430 may enter the slots 445. Additionally, the flanges 440 may deform back to an original shape, or pop back out, upon entering grooves 435 in the outer cover 425, the inner cover 405, or both (once fully inserted into the slots 445). For example, the outer cover 425 may include a groove 435-c (e.g., aligned with the slot 445-a) and a groove 435-e (e.g., aligned with the slot 445-b), and the inner cover 405 may include a groove 435-d (e.g., aligned with the slot 445-a) and a groove 435-f (e.g., aligned with the slot 445-b). As such, once the side cover 430-c is inserted into the slot 445-a, the flange 440-a may engage (e.g., pop out or release into) the groove 435-a and the flange 440-b may engage the groove 435-d. Similarly, once the side cover 430-d is inserted into the slot 445-b, the flange 440-c may engage the groove 435-e and the flange 440-d may engage the groove 435-f. As such, engagement of the flanges 440 with the grooves 435 may result in generation of a force between the side covers 430 (e.g., flanges of the side covers 430) and the outer cover 425 and the inner cover 405, securing the side covers 430 in place between the outer cover 425 and the inner cover 405. Additionally, engagement of the flanges 440 into the grooves 435 may lock the side covers 430 in the slots 445, such that the side covers 430 may not “pop out” or release from the slots 445.

Additionally, or alternatively, as described with reference to the cross-sectional view 400-d and the cross-section view 400-e in FIG. 4C, a side cover 430-e may be inserted, or pressed, into the slot 445-a and a side cover 430-f may be inserted, or pressed, into the slot 445-b. In such cases, the side cover 430-e and the side cover 430-f may each include (e.g., have) one or more chamfers 450 (e.g., sliding surfaces, angled edges, guiding features). For example, the side cover 430-e may include a chamfer 450-a, a chamfer 450-b, a chamfer 450-c, and a chamfer 450-d. In such cases, the chamfer 450-a and the chamfer 450-b may be on an external side of the side cover 430-e (e.g., a top side of the side cover 430-e). Conversely, the chamfer 450-c and the chamfer 450-d may be on an internal side of the side cover 430-a (e.g., a bottom side of the side cover 430-e). That is, the chamfer 450-c and the chamfer 450-d may be on a side of the side cover 430-e that at least partially contacts (e.g., faces) the filler 415. The side cover 430-f may include a similar set of 4 chamfers 450.

Additionally, the outer cover 425, the inner cover 405, or both, may have one or more chamfers 450 in each slot 445, where the one or more chamfers 450 of the outer cover 425, the inner cover 405, or both, are configured to contact at least a subset of the one or more chamfers of the side covers 430 (e.g., one or more chamfers 450 on the internal sides of the side covers 430) during insertion to guide the side covers 430 into the slots 445. For example, the outer cover 425 may include a chamfer 450-e located at least partially in the slot 445-a and the inner cover 405 may include a chamfer 450-f located at least partially in the slot 445-a. Thus, during insertion, the chamfer 450-c of the side cover 430-e may at least partially contact the chamfer 450-e of the outer cover 425 and the chamfer 450-d of the side cover 430-e may at least partially contact the chamfer 450-f of the inner cover 405 to guide the side cover 430-e into the slot 445-a. The outer cover 425 and the inner cover 405 may include similar chamfers 450 located at least partially in the slot 445-b (e.g., that interact with a subset of the set of 4 chamfers 450 of the side cover 430-f during insertion).

In such cases, the chamfers 450 in the side covers 450, the outer cover 425, the inner cover 405, or any combination thereof, may enable the side covers to be inserted into the slots 445 with less force (e.g., as compared to side covers 430 without chamfers 450). Additionally, or alternatively, the chamfers 450 in the side covers 450, the outer cover 425, the inner cover 405, or any combination thereof, may enable the side covers 430 to be inserted into the slots 445 without (e.g., with reduced, with less than a threshold amount of) twisting or unintentional deformation, resulting in an increased force (e.g., stronger hold, tighter fit) between the side covers 430 and walls of the slots 445 (e.g., the outer cover 425 and the inner cover 405) as compared to side covers 430 without chamfers 450.

In some cases, as described previously, at least a portion of the side covers 430 (e.g., a widest dimension of the side covers) may be wider than at least a portion of the slots 445. For example, a widest portion of the side covers 430 may be 0.69 mm while a narrowest portion of the slots 445 may be 0.63 mm. Additionally, or alternatively, the chamfers 450 of the side covers 430, the outer cover 425, the inner cover 405, or any combination thereof, may be small enough (e.g., below a threshold dimension) such that the chamfers 450 may not be visible to the human eye when inserted in the slots 445. For example, the chamfers 450 of the side covers 430, the outer cover 425, the inner cover 405, or any combination thereof, may be associated with a 45 degree angle and may be 0.05 mm. In such cases, the chamfers 450 on external sides of the side covers 430 (e.g., such as the chamfer 450-a and the chamfer 450-b) may enable an outer surface of the ring 104 to feel smooth to the touch (e.g., rather than feeling a ridge or edge wear a side cover 430 without chamfers 450 contacts the outer cover 425 and the inner cover 405).

Though depicted in the context of chamfers 450 on both sides of the side covers 430, this is not to be regarded as a limitation of the present disclosure. In this regard, each side cover 430 may include one or more chamfers 430 on any side or combination of sides (e.g., on each edge, or combination of edges). However, side covers 430 including chamfers 450 on both sides, as depicted in FIG. 4C, may enable a side cover 430 to be inserted into a slot 445 on either side (e.g., reducing manufacturing complexity, as compared to chamfers 450 on one side of the side covers 430). Additionally, or alternatively, though depicted in the context of chamfers 450 on both the outer cover 425 and the inner cover 405, this is not to be regarded as a limitation of the present disclosure. In this regard, the outer cover 425, the inner cover 405, both, or neither, may include one or more chamfers 450.

In some examples, as described with reference to FIGS. 4A, 4B, and 4C, heat may be applied to the ring 104 to further deform the side covers 430 into the grooves 435. That is, upon being inserted into the slots 445, space, or gaps, may still exist between the side covers 430 and the grooves 435. As such, heat may be applied directly to the side covers 430, through the outer cover 425, through the inner cover 405, or any combination thereof, to cause the side covers 430 to deform (e.g., melt) into the grooves 435. In other words, the deformation of the side covers 430 due to heat application may cause the side covers 430 to fill the spaces, or gaps, between the side covers 430 and the grooves 435. In such cases, the side covers 430 may include additional material to enable the deformation due to heat to occur. In other words, while inserted in the slots 445, prior to heat application, a portion of the side covers 430 may extend past surfaces of the inner cover 405 and the outer cover 425 (e.g., creating a bump in a surface of the ring 104). However, as described previously, upon heat application, the side covers 430 may deform into the grooves 435, such that the side covers 430 become flush with the outer cover 425 and the inner cover 405. In other words, the portion of the side covers 430 the previously extended past surfaces of the inner cover 405 and the outer cover 425 may be eliminated (e.g., or reduced, creating a smooth surface of the ring 104).

In some examples, as described with reference to FIGS. 4A, 4B, and 4C, the side covers 430 may be removable to enable the outer cover 425 to be changed or replaced. For example, a removal tool may be inserted into one or more features of the side covers 430, into the slots 445, or both, and maneuvered in a manner that causes the side covers 430 to release from the slots 445 (e.g., and release from the grooves 435). As such, a user 102 may remove the outer cover 425 from around the inner cover 405 and place a new outer cover 425 around the inner cover 405. For example, the user 102 may switch a design of the outer cover 415. That is, the user 102 may remove a first outer cover 425 from around the inner cover 405 (e.g., after removing the side covers 430), where the first outer cover 415 is associated with a first design, and may place a second (e.g., new) outer cover 425, associated with a second (e.g., new) design, around the inner cover 405. In such cases, the first and second designs may differ based on one or more physical features (e.g., ridges, grooves, bumps, texture, material, etc.), one or more visual features (e.g., color, pattern, picture, etc.) or both. Additionally, the user 102 may insert side covers 430 (e.g., new side covers 430 or the same side covers 430 removed from the ring 104) back into the slots 445 between the new outer cover 425 and the inner cover 405. In such cases, the user 102 may utilize one or more pressing tools to insert the side covers 430 back into the slots 445. In some examples, removal of the side covers 430 and the outer cover 425 may enable one or more components of the ring 104 to be replaced, repaired, or both (e.g., in the cases of failure). Additionally, or alternatively, the user 102 may remove the outer cover 425 from around the inner cover 405 and place the outer cover 425 over a new ring engine assembly (e.g., a new inner cover 405 with attached components).

Though described in the context of multiple techniques for securing the outer cover 425 to the inner cover 405, this is not to be regarded as a limitation of the present disclosure. That is, any combination of the techniques described herein may be employed to secure, or lock, an outer cover 425 of a ring 104 to an inner cover 405 of the ring 104. Conversely, any technique described herein may be employed in a standalone manner to secure, or lock, an outer cover 425 of a ring 104 to an inner cover 405 of the ring 104.

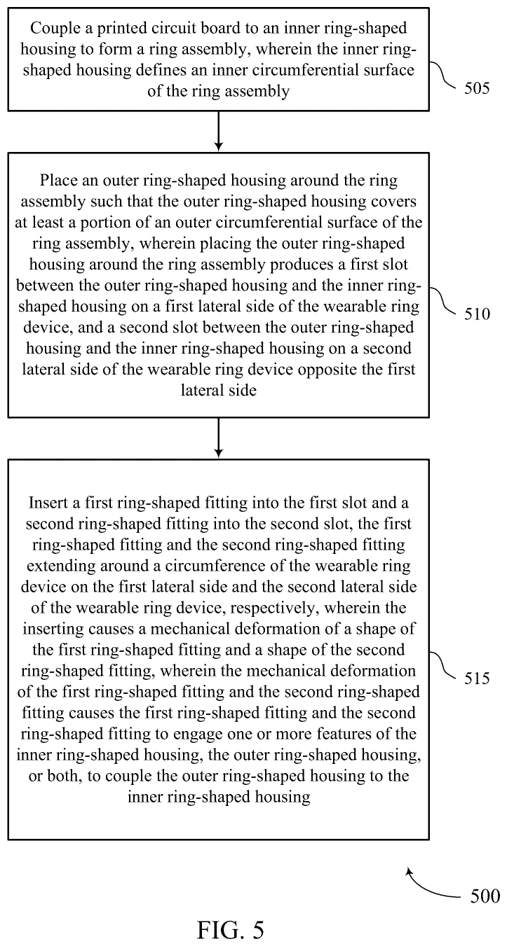

FIG. 5 shows a flowchart illustrating a method 500 that supports techniques for fixing an outer cover to a wearable ring device in accordance with aspects of the present disclosure. The operations of the method 500 may be implemented by a wearable device or its components as described herein. For example, the operations of the method 500 may be performed by a wearable device as described with reference to FIGS. 1 through 5. In some examples, a wearable device may execute a set of instructions to control the functional elements of the wearable device to perform the described functions. Additionally, or alternatively, the wearable device may perform aspects of the described functions using special-purpose hardware.

At 505, the method may include coupling a PCB to an inner ring-shaped housing to form a ring assembly, wherein the inner ring-shaped housing defines an inner circumferential surface of the ring assembly. The operations of block 505 may be performed in accordance with examples as disclosed herein.

At 510, the method may include placing an outer ring-shaped housing around the ring assembly such that the outer ring-shaped housing covers at least a portion of an outer circumferential surface of the ring assembly, wherein placing the outer ring-shaped housing around the ring assembly produces a first slot between the outer ring-shaped housing and the inner ring-shaped housing on a first lateral side of the wearable ring device, and a second slot between the outer ring-shaped housing and the inner ring-shaped housing on a second lateral side of the wearable ring device opposite the first lateral side. The operations of block 510 may be performed in accordance with examples as disclosed herein.

At 515, the method may include inserting a first ring-shaped fitting into the first slot and a second ring-shaped fitting into the second slot, the first ring-shaped fitting and the second ring-shaped fitting extending around a circumference of the wearable ring device on the first lateral side and the second lateral side of the wearable ring device, respectively, wherein the inserting causes a mechanical deformation of a shape of the first ring-shaped fitting and a shape of the second ring-shaped fitting, wherein the mechanical deformation of the first ring-shaped fitting and the second ring-shaped fitting causes the first ring-shaped fitting and the second ring-shaped fitting to engage one or more features of the inner ring-shaped housing, the outer ring-shaped housing, or both, to couple the outer ring-shaped housing to the inner ring-shaped housing. The operations of block 515 may be performed in accordance with examples as disclosed herein.

It should be noted that the methods described above describe possible implementations, and that the operations and the steps may be rearranged or otherwise modified and that other implementations are possible. Furthermore, aspects from two or more of the methods may be combined.

A wearable ring device (e.g., apparatus) is described. The wearable ring device may include an inner ring-shape housing comprising one or more apertures, wherein the inner ring-shaped housing defines an inner circumferential surface of the wearable ring device; one or more sensors dispose at least partially within the inner ring-shaped housing, the one or more sensors configured to acquire physiological data from a user through the one or more apertures; an outer ring-shape housing that at least partially surrounds the inner ring-shaped housing, wherein the outer ring-shaped housing defines an outer circumferential surface of the wearable ring device; a first ring-shape fitting at least partially disposed within a first slot between the outer ring-shaped housing and the inner ring-shaped housing on a first lateral side of the wearable ring device; and a second ring-shape fitting at least partially disposed within a second slot between the outer ring-shaped housing and the inner ring-shaped housing on a second lateral side of the wearable ring device, wherein the first ring-shaped fitting and the second ring-shaped fitting are configured to undergo a mechanical deformation upon being inserted into the first slot and the second slot, respectively, wherein the mechanical deformation of the first ring-shaped fitting and the second ring-shaped fitting causes the first ring-shaped fitting and the second ring-shaped fitting to engage one or more features of the inner ring-shaped housing, the outer ring-shaped housing, or both, to couple the outer ring-shaped housing to the inner ring-shaped housing.

In some examples, the wearable ring device may include one or more first chamfers in the outer ring-shaped housing, the inner ring-shaped housing, or both, and one or more second chamfers in each of the first ring-shaped fitting and the second ring-shaped fitting, wherein the one or more first chamfers are configured to contact at least a subset the one or more second chamfers to guide the first ring-shaped fitting and the second ring-shaped fitting into the first slot and the second slot, respectively.

In some examples, the one or more second chamfers may include a first set of chamfers on a top side of each of the first ring-shaped fitting and the second ring-shaped fitting, and a second set of chamfers on a bottom side of each of the first ring-shaped fitting and the second ring-shaped fitting.

In some examples, the one or more first chamfers in the outer ring-shaped housing, the inner ring-shaped housing, or both, may include a first set of chamfers at least partially within the first slot and a second set of chamfers at least partially within the second slot

In some examples, the wearable ring device may include one or more first grooves in the outer ring-shaped housing, the inner ring-shaped housing, or both, wherein the one or more first grooves may be located at least partially in the first slot and one or more second grooves in the outer ring-shaped housing, the inner ring-shaped housing, or both, wherein the one or more second grooves may be located at least partially in the second slot, wherein the one or more features comprise the one or more first grooves and the one or more second grooves, wherein the first ring-shaped fitting may be configured to deform into the one or more first grooves, and the second ring-shaped fitting may be configured to deform into the one or more second grooves.

In some examples, the wearable ring device may include, a first set of flanges on the first ring-shaped fitting, wherein the first set of flanges may be configured to undergo the mechanical deformation from a first shape to a second shape during insertion of the first ring-shaped fitting into the first slot, wherein the first set of flanges may be configured to undergo a second mechanical deformation from the second shape back to the first shape after insertion into the first slot, wherein the second mechanical deformation causes the first set of flanges to engage the one or more first grooves and a second set of flanges on the second ring-shaped fitting, wherein the second set of flanges may be configured to undergo the mechanical deformation from the first shape to the second shape during insertion of the second ring-shaped fitting into the second slot, wherein the second set of flanges may be configured to undergo a third mechanical deformation from the second shape back to the first shape after insertion into the second slot, wherein the third mechanical deformation causes the second set of flanges to engage the one or more second grooves.

In some examples, the first set of flanges on the first ring-shaped fitting may extend wider than a width of the first slot, and the second set of flanges on the second ring-shaped fitting may extend wider than a width of the second slot.

In some examples, the first ring-shaped fitting and the second ring-shaped fitting may be configured to undergo the mechanical deformation under application of heat after being inserted into the first slot and the second slot respectively and the application of heat may cause the first ring-shaped fitting and the second ring-shaped fitting to deform at least partially into a shape of the one or more first grooves and a shape of the one or more second grooves, respectively.

In some examples, a thickness of the first ring-shaped fitting may be wider than the first slot and a thickness of the second ring-shaped fitting may be wider than the second slot.

In some examples, the wearable ring device may include a binding agent positioned between the outer ring-shaped housing and the inner ring-shaped housing, wherein the outer ring-shaped housing may be coupled to the inner ring-shaped housing based at least in part on the binding agent.

In some examples, the binding agent comprises the binding agent comprises heat or light-activated glue configured to cure under application of heat or light, respectively, through the first ring-shaped fitting, the second ring-shaped fitting, or both, and wherein the outer ring-shaped housing is coupled to the inner ring-shaped housing based at least in part on curing the heat or light-activated glue.

In some examples, the binding agent comprises a heat activated film configured to activate under application of heat through the outer ring-shaped housing, the inner ring-shaped housing, the first ring-shaped fitting, the second ring-shaped fitting, or any combination thereof and the outer ring-shaped housing may be coupled to the inner ring-shaped housing based at least in part on activating the heat activated film.

In some examples, the inner ring-shaped housing, the outer ring-shaped housing, or both, comprise a textured surfaces that may be configured to engage with a textured surface of the first ring-shaped fitting, the second ring-shaped fitting, or both.

In some examples, the first ring-shaped fitting and the second ring-shaped fitting may be configured to be removed via insertion of a removal tool at least partially into the first slot and the second slot, respectively.

In some examples, the inner ring-shaped housing and the outer ring-shaped housing comprise a same material or different materials.

In some examples, the first ring-shaped fitting and the second ring-shaped fitting comprise one or more non-metal materials that may be configured to enable wireless signals to propagate into and out of the wearable ring device through the first ring-shaped fitting and the second ring-shaped fitting, respectively.

In some examples, coupling the outer ring-shaped housing to the inner ring-shaped housing via insertion of the first ring-shaped fitting into the first slot and the second ring-shaped fitting into the second slot creates a waterproof seal between the outer ring-shaped housing and the inner ring-shaped housing.

In some examples, the wearable ring device may include a filler material that may be injected into a cavity between the inner ring-shaped housing and a surface of a mold to fill at least a portion of the one or more apertures of the inner ring-shaped housing, wherein the filler material may be further configured to bind a PCB to the inner ring-shaped housing.

A method for manufacturing a wearable ring device by an apparatus is described. The method may include coupling a PCB to an inner ring-shaped housing to form a ring assembly, wherein the inner ring-shaped housing defines an inner circumferential surface of the ring assembly, placing an outer ring-shaped housing around the ring assembly such that the outer ring-shaped housing covers at least a portion of an outer circumferential surface of the ring assembly, wherein placing the outer ring-shaped housing around the ring assembly produces a first slot between the outer ring-shaped housing and the inner ring-shaped housing on a first lateral side of the wearable ring device, and a second slot between the outer ring-shaped housing and the inner ring-shaped housing on a second lateral side of the wearable ring device opposite the first lateral side, and inserting a first ring-shaped fitting into the first slot and a second ring-shaped fitting into the second slot, the first ring-shaped fitting and the second ring-shaped fitting extending around a circumference of the wearable ring device on the first lateral side and the second lateral side of the wearable ring device, respectively, wherein the inserting causes a mechanical deformation of a shape of the first ring-shaped fitting and a shape of the second ring-shaped fitting, wherein the mechanical deformation of the first ring-shaped fitting and the second ring-shaped fitting causes the first ring-shaped fitting and the second ring-shaped fitting to engage one or more features of the inner ring-shaped housing, the outer ring-shaped housing, or both, to couple the outer ring-shaped housing to the inner ring-shaped housing.

An apparatus for manufacturing a wearable ring device is described. The apparatus may include one or more memories storing processor executable code, and one or more processors coupled with the one or more memories. The one or more processors may individually or collectively operable to execute the code to cause the apparatus to couple a PCB to an inner ring-shaped housing to form a ring assembly, wherein the inner ring-shaped housing defines an inner circumferential surface of the ring assembly, place an outer ring-shaped housing around the ring assembly such that the outer ring-shaped housing covers at least a portion of an outer circumferential surface of the ring assembly, wherein placing the outer ring-shaped housing around the ring assembly produces a first slot between the outer ring-shaped housing and the inner ring-shaped housing on a first lateral side of the wearable ring device, and a second slot between the outer ring-shaped housing and the inner ring-shaped housing on a second lateral side of the wearable ring device opposite the first lateral side, and insert a first ring-shaped fitting into the first slot and a second ring-shaped fitting into the second slot, the first ring-shaped fitting and the second ring-shaped fitting extending around a circumference of the wearable ring device on the first lateral side and the second lateral side of the wearable ring device, respectively, wherein the inserting causes a mechanical deformation of a shape of the first ring-shaped fitting and a shape of the second ring-shaped fitting, wherein the mechanical deformation of the first ring-shaped fitting and the second ring-shaped fitting causes the first ring-shaped fitting and the second ring-shaped fitting to engage one or more features of the inner ring-shaped housing, the outer ring-shaped housing, or both, to couple the outer ring-shaped housing to the inner ring-shaped housing.

Another apparatus for manufacturing a wearable ring device is described. The apparatus may include means for coupling a PCB to an inner ring-shaped housing to form a ring assembly, wherein the inner ring-shaped housing defines an inner circumferential surface of the ring assembly, means for placing an outer ring-shaped housing around the ring assembly such that the outer ring-shaped housing covers at least a portion of an outer circumferential surface of the ring assembly, wherein placing the outer ring-shaped housing around the ring assembly produces a first slot between the outer ring-shaped housing and the inner ring-shaped housing on a first lateral side of the wearable ring device, and a second slot between the outer ring-shaped housing and the inner ring-shaped housing on a second lateral side of the wearable ring device opposite the first lateral side, and means for inserting a first ring-shaped fitting into the first slot and a second ring-shaped fitting into the second slot, the first ring-shaped fitting and the second ring-shaped fitting extending around a circumference of the wearable ring device on the first lateral side and the second lateral side of the wearable ring device, respectively, wherein the inserting causes a mechanical deformation of a shape of the first ring-shaped fitting and a shape of the second ring-shaped fitting, wherein the mechanical deformation of the first ring-shaped fitting and the second ring-shaped fitting causes the first ring-shaped fitting and the second ring-shaped fitting to engage one or more features of the inner ring-shaped housing, the outer ring-shaped housing, or both, to couple the outer ring-shaped housing to the inner ring-shaped housing.

A non-transitory computer-readable medium storing code for manufacturing a wearable ring device is described. The code may include instructions executable by a processor to couple a PCB to an inner ring-shaped housing to form a ring assembly, wherein the inner ring-shaped housing defines an inner circumferential surface of the ring assembly, place an outer ring-shaped housing around the ring assembly such that the outer ring-shaped housing covers at least a portion of an outer circumferential surface of the ring assembly, wherein placing the outer ring-shaped housing around the ring assembly produces a first slot between the outer ring-shaped housing and the inner ring-shaped housing on a first lateral side of the wearable ring device, and a second slot between the outer ring-shaped housing and the inner ring-shaped housing on a second lateral side of the wearable ring device opposite the first lateral side, and insert a first ring-shaped fitting into the first slot and a second ring-shaped fitting into the second slot, the first ring-shaped fitting and the second ring-shaped fitting extending around a circumference of the wearable ring device on the first lateral side and the second lateral side of the wearable ring device, respectively, wherein the inserting causes a mechanical deformation of a shape of the first ring-shaped fitting and a shape of the second ring-shaped fitting, wherein the mechanical deformation of the first ring-shaped fitting and the second ring-shaped fitting causes the first ring-shaped fitting and the second ring-shaped fitting to engage one or more features of the inner ring-shaped housing, the outer ring-shaped housing, or both, to couple the outer ring-shaped housing to the inner ring-shaped housing.

In some examples of the method, apparatus, and non-transitory computer-readable medium described herein, the outer ring-shaped housing, the inner ring-shaped housing, or both, may include one or more first chamfers, and each of the first ring-shaped fitting and the second ring-shaped fitting may include one or more second chamfers, such that inserting the first ring-shaped fitting into the first slot and the second ring-shaped fitting into the second slot may be based on the one or more first chamfers contacting at least a subset of the one or more second chamfers and guiding the the first ring-shaped fitting and the second ring-shaped fitting into the first slot and the second slot, respectively

In some examples of the method, apparatus, and non-transitory computer-readable medium described herein, the one or more features of the inner ring-shaped housing, the outer ring-shaped housing, or both, comprise one or more first grooves located at least partially in the first slot and one or more second grooves located at least partially in the second slot, the one or more features comprise the one or more first grooves and the one or more second grooves, inserting the first ring-shaped fitting causes the first ring-shaped fitting to deform into the one or more first grooves, and inserting the second ring-shaped fitting causes the second ring-shaped fitting to deform into the one or more second grooves.

Some examples of the method, apparatus, and non-transitory computer-readable medium described herein may further include operations, features, means, or instructions for applying heat to the first ring-shaped fitting and the second ring-shaped fitting after inserting the first ring-shaped fitting and the second ring-shaped fitting into the first slot and second slot, respectively, wherein applying heat to the first ring-shaped fitting and the second ring-shaped fitting causes the first ring-shaped fitting and the second ring-shaped fitting to deform at least partially into a shape of the one or more first grooves and a shape of the one or more second grooves, respectively.

Some examples of the method, apparatus, and non-transitory computer-readable medium described herein may further include operations, features, means, or instructions for inserting a heat or light-activated glue between the outer ring-shaped housing and the inner ring-shaped housing and applying heat or light, respectively, to the heat or light-activated glue through the first ring-shaped fitting, the second ring-shaped fitting, or both, to cure the heat or light-activated glue, wherein coupling the outer ring-shaped housing to the inner ring-shaped housing may be based at least in part on curing the heat or light-activated glue.

Some examples of the method, apparatus, and non-transitory computer-readable medium described herein may further include operations, features, means, or instructions for inserting a heat activated film between the outer ring-shaped housing and the inner ring-shaped housing and applying heat to the heat activated film through the outer ring-shaped housing, the inner ring-shaped housing, the first ring-shaped fitting, the second ring-shaped fitting, or any combination thereof, to activate the heat activated film, wherein coupling the outer ring-shaped housing to the inner ring-shaped housing may be based at least in part on activating the heat activated film.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
an inner ring-shaped housing comprising one or more apertures, wherein the inner ring-shaped housing defines an inner circumferential surface of the wearable ring device;
one or more sensors disposed at least partially within the inner ring-shaped housing, the one or more sensors configured to acquire physiological data from a user through the one or more apertures;
an outer ring-shaped housing that at least partially surrounds the inner ring-shaped housing, wherein the outer ring-shaped housing defines an outer circumferential surface of the wearable ring device;
a first ring-shaped fitting at least partially disposed within a first slot between the outer ring-shaped housing and the inner ring-shaped housing on a first lateral side of the wearable ring device; and
a second ring-shaped fitting at least partially disposed within a second slot between the outer ring-shaped housing and the inner ring-shaped housing on a second lateral side of the wearable ring device, wherein the first ring-shaped fitting and the second ring-shaped fitting are configured to undergo a mechanical deformation upon being inserted into the first slot and the second slot, respectively, wherein the mechanical deformation of the first ring-shaped fitting and the second ring-shaped fitting causes the first ring-shaped fitting and the second ring-shaped fitting to engage one or more features of the inner ring-shaped housing, the outer ring-shaped housing, or both, to couple the outer ring-shaped housing to the inner ring-shaped housing.

2. The wearable ring device of claim 1, further comprising:
one or more first chamfers in the outer ring-shaped housing, the inner ring-shaped housing, or both; and
one or more second chamfers in each of the first ring-shaped fitting and the second ring-shaped fitting, wherein the one or more first chamfers are configured to contact at least a subset the one or more second chamfers to guide the first ring-shaped fitting and the second ring-shaped fitting into the first slot and the second slot, respectively.

3. The wearable ring device of claim 2, wherein the one or more second chamfers comprise a first set of chamfers on a top side of each of the first ring-shaped fitting and the second ring-shaped fitting and a second set of chamfers on a bottom side of each of the first ring-shaped fitting and the second ring-shaped fitting.

4. The wearable ring device of claim 2, wherein the one or more first chamfers in the outer ring-shaped housing, the inner ring-shaped housing, or both, comprise a first set of chamfers at least partially within the first slot and a second set of chamfers at least partially within the second slot.

5. The wearable ring device of claim 1, further comprising:
one or more first grooves in the outer ring-shaped housing, the inner ring-shaped housing, or both, wherein the one or more first grooves are located at least partially in the first slot; and
one or more second grooves in the outer ring-shaped housing, the inner ring-shaped housing, or both, wherein the one or more second grooves are located at least partially in the second slot, wherein the one or more features comprise the one or more first grooves and the one or more second grooves, wherein the first ring-shaped fitting is configured to deform into the one or more first grooves, and the second ring-shaped fitting is configured to deform into the one or more second grooves.

6. The wearable ring device of claim 5, further comprising:
a first set of flanges on the first ring-shaped fitting, wherein the first set of flanges are configured to undergo the mechanical deformation from a first shape to a second shape during insertion of the first ring-shaped fitting into the first slot, wherein the first set of flanges are configured to undergo a second mechanical deformation from the second shape back to the first shape after insertion into the first slot, wherein the second mechanical deformation causes the first set of flanges to engage the one or more first grooves; and
a second set of flanges on the second ring-shaped fitting, wherein the second set of flanges are configured to undergo the mechanical deformation from the first shape to the second shape during insertion of the second ring-shaped fitting into the second slot, wherein the second set of flanges are configured to undergo a third mechanical deformation from the second shape back to the first shape after insertion into the second slot, wherein the third mechanical deformation causes the second set of flanges to engage the one or more second grooves.

7. The wearable ring device of claim 5, wherein the first ring-shaped fitting and the second ring-shaped fitting are configured to undergo the mechanical deformation under application of heat after being inserted into the first slot and the second slot respectively, and wherein the application of heat causes the first ring-shaped fitting and the second ring-shaped fitting to deform at least partially into a shape of the one or more first grooves and a shape of the one or more second grooves, respectively.

8. The wearable ring device of claim 1, wherein a thickness of the first ring-shaped fitting is wider than the first slot and a thickness of the second ring-shaped fitting is wider than the second slot.

9. The wearable ring device of claim 1, further comprising:
a binding agent positioned between the outer ring-shaped housing and the inner ring-shaped housing, wherein the outer ring-shaped housing is coupled to the inner ring-shaped housing based at least in part on the binding agent.

10. The wearable ring device of claim 8, wherein the binding agent comprises heat or light-activated glue configured to cure under application of heat or light, respectively, through the first ring-shaped fitting, the second ring-shaped fitting, or both, and wherein the outer ring-shaped housing is coupled to the inner ring-shaped housing based at least in part on curing the heat or light-activated glue.

11. The wearable ring device of claim 8, wherein the binding agent comprises a heat activated film configured to activate under application of heat through the outer ring-shaped housing, the inner ring-shaped housing, the first ring-shaped fitting, the second ring-shaped fitting, or any combination thereof, and wherein the outer ring-shaped housing is coupled to the inner ring-shaped housing based at least in part on activating the heat activated film.

12. The wearable ring device of claim 1, wherein the inner ring-shaped housing, the outer ring-shaped housing, or both, comprise a textured surfaces that is configured to engage with a textured surface of the first ring-shaped fitting, the second ring-shaped fitting, or both.

13. The wearable ring device of claim 1, wherein the first ring-shaped fitting and the second ring-shaped fitting are configured to be removed via insertion of a removal tool at least partially into the first slot and the second slot, respectively.

14. The wearable ring device of claim 1, wherein the first ring-shaped fitting and the second ring-shaped fitting comprise one or more non-metal materials that are configured to enable wireless signals to propagate into and out of the wearable ring device through the first ring-shaped fitting and the second ring-shaped fitting, respectively.

15. The wearable ring device of claim 1, wherein coupling the outer ring-shaped housing to the inner ring-shaped housing via insertion of the first ring-shaped fitting into the first slot and the second ring-shaped fitting into the second slot creates a waterproof seal between the outer ring-shaped housing and the inner ring-shaped housing.

16. The wearable ring device of claim 1, further comprising:
a filler material that is injected into a cavity between the inner ring-shaped housing and a surface of a mold to fill at least a portion of the one or more apertures of the inner ring-shaped housing, wherein the filler material is further configured to bind a printed circuit board to the inner ring-shaped housing.

17. A method for manufacturing a wearable ring device, comprising:
coupling a printed circuit board to an inner ring-shaped housing to form a ring assembly, wherein the inner ring-shaped housing defines an inner circumferential surface of the ring assembly;
placing an outer ring-shaped housing around the ring assembly such that the outer ring-shaped housing covers at least a portion of an outer circumferential surface of the ring assembly, wherein placing the outer ring-shaped housing around the ring assembly produces a first slot between the outer ring-shaped housing and the inner ring-shaped housing on a first lateral side of the wearable ring device, and a second slot between the outer ring-shaped housing and the inner ring-shaped housing on a second lateral side of the wearable ring device opposite the first lateral side; and
inserting a first ring-shaped fitting into the first slot and a second ring-shaped fitting into the second slot, the first ring-shaped fitting and the second ring-shaped fitting extending around a circumference of the wearable ring device on the first lateral side and the second lateral side of the wearable ring device, respectively, wherein the inserting causes a mechanical deformation of a shape of the first ring-shaped fitting and a shape of the second ring-shaped fitting, wherein the mechanical deformation of the first ring-shaped fitting and the second ring-shaped fitting causes the first ring-shaped fitting and the second ring-shaped fitting to engage one or more features of the inner ring-shaped housing, the outer ring-shaped housing, or both, to couple the outer ring-shaped housing to the inner ring-shaped housing.

18. The method of claim 17, wherein the outer ring-shaped housing, the inner ring-shaped housing, or both, comprise one or more first chamfers, wherein each of the first ring-shaped fitting and the second ring-shaped fitting comprise one or more second chamfers, and wherein inserting the first ring-shaped fitting into the first slot and the second ring-shaped fitting into the second slot is based at least in part on the one or more first chamfers contacting at least a subset of the one or more second chamfers and guiding the the first ring-shaped fitting and the second ring-shaped fitting into the first slot and the second slot, respectively.

19. The method of claim 17, wherein the one or more features of the inner ring-shaped housing, the outer ring-shaped housing, or both, comprise one or more first grooves located at least partially in the first slot and one or more second grooves located at least partially in the second slot, wherein inserting the first ring-shaped fitting causes the first ring-shaped fitting to deform into the one or more first grooves, and wherein inserting the second ring-shaped fitting causes the second ring-shaped fitting to deform into the one or more second grooves.

20. The method of claim 17, further comprising:
inserting a heat or light-activated glue between the outer ring-shaped housing and the inner ring-shaped housing; and
applying heat or light, respectively, to the heat or light-activated glue through the first ring-shaped fitting, the second ring-shaped fitting, or both, to cure the heat or light-activated glue, wherein coupling the outer ring-shaped housing to the inner ring-shaped housing is based at least in part on curing the heat or light-activated glue.
